(19) United States
(12) Patent Application Publication
(10) Pub. No.: US 2005/0181020 A1
(43) Pub. Date: Aug. 18, 2005

(54) Edible film formulation

(76) Inventor: Reg Macquarrie, Barrie (CA)

Correspondence Address: SHLESINGER, ARKWRIGHT & GARVEY LLP, 3000 South Eads Street, Arlington, VA, 22202, US

(21) Appl. No.: 11/043,910

(22) Filed: Jan. 13, 2005

Related U.S. Application Data
(63) Continuation of application No. 10/024,584, filed on Dec. 21, 2001, now abandoned.
(60) Provisional application No. 60/257,100, filed on Dec. 22, 2000.

(51) Int. Cl. A61K 47/00; A23G 3/00

(52) U.S. Cl. 424/439; 426/660

(57) ABSTRACT

Edible films incorporated carrageenan in conjunction with insoluble and inert carbohydrate components, such as high-amylose starch, have been found to exhibit superior properties as material for meat casings, including high strength and excellent adhesion to the meat. Liquid compositions for casting into such edible films are disclosed which facilitate the efficient production of sausage and other film-encased meat products using conventional forming apparatus.

### RELATED APPLICATIONS

This application is a replacement of provisional application No. 60/257,100 filed on Dec. 22, 2000 and entitled Edible Food Formulation.

### BACKGROUND OF THE INVENTION

In my International patent application No. PCT/CA00/00565 published on Nov. 16, 2000 under International publication No. WO 00/67582, I describe carrageenan-based films useful as substitutes for conventional edible collagen films of the kind employed in ham and sausage production. Edible films such as sausage casings having superior properties were cast from liquid compositions comprising, along with carrageenan and water, suitable non-thermoreversible gel-forming polymers, preferably, Konjac and gellan gums.

### DESCRIPTION OF THE INVENTION

The present invention is directed to different carrageenan-based film-forming liquid compositions for the preparation of meat films and casings, characterized by the incorporation in the composition of an insoluble, inert carbohydrate component which is dispersed throughout the carrageenan film web.

Specifically, I have discovered that the combination of carrageenan and a suitable insoluble carbohydrate (together with water, glycerol and, optionally, galactomann or other gum) has a significant effect on the properties and processability of the resulting edible film. It may be that the incorporation of the non-soluble fraction affects the way in which the dried carrageenan web absorbs moisture, which in term results in the observed salutary properties of the film.

An example of a formulation of film according to the present invention is as follows:

| Component | Percentage Range |
|---|---|
| Carrageenan | 25%-45% |
| Galactomann | 0%-10% |
| Insoluble carbohydrate | 3%-30% |
| Glycerol or other polyol | 10%-40% |
| Water | 10%-35% |

The following table affords a qualitative comparison of films according to the present invention, which include the insoluble starch component, to similar films in which the principal film-forming agent is again, carrageenan but no starch is present (insoluble carbohydrate):

| | | |
|---|---|---|
| | Starch + | Carrageenan |
| Film type | Carrageenan | only |

Film Properties

| | | |
|---|---|---|
| Dry strength: | Very good | Good |
| Wet Strength: | Very good | Acceptable |
| Adhesion to meat: | Excellent | Poor |
| Melting point: | >100% humidity | 65% rH |
| Solubility: | Poor | Good |

Processing Character

| | | |
|---|---|---|
| Castability: | Excellent | Difficult |
| Belt release: | Good | Difficult |
| Drying: | Quicker | Slower |

The insoluble-inert-component could be one or more of: high amylose starch, cellulose components, naturally insoluble gums, fibrous components of plant materials, beta and other glucans. An important consideration in the selection of this component that it be hypoallergenic.

I have observed that the incorporation of insoluble components also results in the following advantages over carrageenan films without starch:

Production Improvements

H. Facilitates easier extrusion of the film by preventing the instant transition of the liquid carrageenan to a gel. There is a reduction in gel formation during extrusion.
I. Incorporation of an insoluble polymer promotes quicker drying of the film by facilitating the release of moisture from the mix.
J. Use of an insoluble component also results in improved film release from the belt after drying and reduces the requirement of coating a belt or using release aids.
   Film Processing Improvements:
A. Films that use an insoluble component exhibit less tendency to absorb moisture from processing environments and are thus more stable.
B. Films with insoluble components-are more rigid and this leads to improved glide of the film over stainless steel forming systems.
C. Films with an insoluble component reduce the tendency of the film to block on a roll. The film shows better release with no adhesion to itself.
   Film Function Improvements
A. Films with an insoluble component dry faster onto a substrate.
B. Pure carrageenan films result in a final product that has a shiny plastic appearance that does not appear natural. Films that utilize some insoluble component tend to have a flatter tone that results in a more natural appearance.

### EXAMPLE 1

| Component | percent by weight |
|---|---|
| Carrageenan | 29 |
| High amylose starch | 20 |
| Locust bean gum | 8 |
| Glycerin | 24 |
| Water | 17 |

This film was very strong with a burst strength of 19 psi and an elongation of 45%. The film also appeared to be slightly opaque.

### EXAMPLE 2

| Component | percent by weight |
|---|---|
| Carrageenan | 45 |
| Cellulose | 5 |
| Tara gum | 7 |
| Glycerin | 20 |
| Water | 23 |

This film was slightly more transparent than the film in Example 1 but exhibited an elongation of about 75%.

### EXAMPLE 3

| Component | percent by weight |
|---|---|
| Carrageenan | 30 |
| Beta Glucan | 8 |
| High amylose starch | 20 |
| Glycerin | 25 |
| Water | 17 |

This film was very similar to the film in Example 1 and in addition exhibited a slight texturing of the surface.

### EXAMPLE 4

| Component | percent by weight |
|---|---|
| Carrageenan | 36 |
| Starch | 9 |
| Locust Bean Gum | 4 |
| Glycerin | 30 |
| Caramel | 5 |
| Water | 15 |

This film had both good strength (17 psi) and elongation (50%). The optional addition of caramel powder resulted in a dark brown colour which is pleasing and desirable for some products.

In the preparation of films according to the present invention, the water component is heated to 85° C., then the inert materials and any gums are added under vacuum with continuous agitation. Glycerin and/or other suitable plasticizing polyols are then added under vacuum and blended with the composition. Finally, the liquid composition is cast on to a belt or to a drum dryer and is dried to form the casing film.

By appropriate and conventional adjustments of the extrusion dryer and the drying belt, the solutions according to present invention may be formed in drying into tubular casings or bags of various sizes. Sheets made from the compositions can be heat sealed to produce pouches and bags for food applications.

The basic film formulation according to the present invention can be augmented with the addition of the following:

| | |
|---|---|
| A. Colours | Natural and artificial |
| B. Flavours | Natural and artificial - smoke flavours |
| C. Spices | Ground or liquid spices can |
| | be added to the base web |
| D. Preservatives | |

A multi-layer film can be produced by applying a second liquid solution to the base film and drying this or by co-laminating a film to the base web. In this process, it is possible to apply colours or flavours by first preparing a slurry of these and then rolling them onto the existing film.

1. A liquid composition for casting into an edible film, comprising uniform mixture of 25 to 45 wt. % of carrageenan, from 3 to 30 wt. % of a substantially water-insoluble edible carbohydrate, from 10 to 40 wt. % of a plasticizing polyol and from to 10 to 35 wt. % of water.

2. A liquid composition according to claim 1, wherein said plasticizing polyol is glycerol.

3. A liquid composition according to claim 2, further comprising up to 10 wt. % of an edible gum.

4. A liquid composition according to claim 3, wherein said substantially water-insoluble water carbohydrate is selected from hypoallergenic members of the group consisting of high-amylose starch, components of cellulose, insoluble gums, fibrous vegetable materials and glucans.

5. A liquid composition according to claim 3, wherein said edible gum is selected from the group consisting of galactomann, locust bean gum and tara gum.

6. A liquid composition according to claim 4, further comprising sufficient caramel powder or other colouring material to impart a desired colour to an edible film prepared from the liquid composition.
